(19) United States
(12) Patent Application Publication
(10) Pub. No.: US 2010/0221304 A1
(43) Pub. Date: Sep. 2, 2010

(54) Bionanocomposite Materials and Methods For Producing and Using the Same

(75) Inventors: Wei Tan, Broomfield, CO (US); Krishna Madhavan, Boulder, CO (US); Walter Bonani, Boulder, CO (US)

Correspondence Address: Don D. Cha, 547 Buena Vista Road, Golden, CO, 80401, US

(73) Assignee: The Regents of the University of Colorado, a body corporate, Denver, CO (US)

(21) Appl. No.: 12/714,162

(22) Filed: Feb. 26, 2010

Related U.S. Application Data
(60) Provisional application No. 61/155,832, filed on Feb. 26, 2009.

(51) Int. Cl. A61F 2/82; B29C 63/18; B29D 23/00; A61P 9/00

(52) U.S. Cl. 424/423; 427/2.3; 428/35.6; 977/702

(57) ABSTRACT

The present invention provides bionanocomposite materials comprising at least two coaxial layers of bionanocomposites, and methods for producing and using the same. The bionanocomposite materials of the present invention comprise a core structure and a shell structure encapsulating the core structure, where one of the core structure or the shell structure comprises a porous biocompatible natural-derived material and the other comprises a biocompatible biomimetic nanostructure.

### CROSS-REFERENCE TO RELATED APPLICATIONS

This application claims the priority benefit of U.S. Provisional Application No. 61/155,832, filed Feb. 26, 2009, which is incorporated herein by reference in its entirety.

### FIELD OF THE INVENTION

The present invention relates to bionanocomposite materials comprising a plurality of coaxial layers and a hollow or porous interior, and methods for producing and using the same. In particular the present invention relates to tubular or tube-like bionanocomposite materials.

### BACKGROUND OF THE INVENTION

Small-diameter (≦6 mm) vascular grafts, man-made materials to replace blood vessels, become a symbol for the limitations of modern biotechnology. These grafts are useful for a variety of medical conditions including, but not limited to, coronary artery grafts, bypass grafts (e.g., for atherosclerosis), pediatric shunt grafts (e.g., for congenital heart diseases), arteriovenous (A/V) grafts (e.g., for vascular access in hemodialysis patients with kidney failure), treatment for aneurism (AAA and TAA), as well as other clinical conditions that can benefit from artificial grafts. Graft-associated dysfunction is the leading cause of morbidity and hospitalization in these patients and costs greater than billions of dollars per annum.

Vascular access is the method used to access the bloodstream for hemodialysis patients. Hemodialysis removes blood from the body and routes it to an artificial kidney machine where the blood is cleansed and returned to the patient. Hemodialysis patients require easy and routine access (e.g., three times a week) to the bloodstream. The most widely used form of long-term vascular access is prosthetic arteriovenous (A/V) graft. A primary material used in the construction of A/V graft is expanded polytetrafluoroethylene (ePTFE). In fact, the ePTFE graft has become a standard among vascular surgeons, and occupies more than 90% of the market. However, the ePTFE graft has at least two major disadvantages. One disadvantage is that it must be allowed to “mature” for at least two weeks after the implant procedure to ensure that sufficient tissue in-growth has occurred. The other disadvantage is its high long-term failure rate, for example, 1-year and 2-year failure rates are approximately 50% and 75%, respectively.

The graft-associated vascular access dysfunction is the most common cause of morbidity and hospitalization in patients with kidney failure. Despite the magnitude of graft-associated problems, currently there are very few effective approaches. The majority of the graft-associated problems are associated with the mechanical and biological properties of ePTFE material. Graft made of ePTFE lacks a cell-compatible and blood-compatible surface and thus requires time to “mature” (to ensure sufficient tissue in-growth) before implantation. Additionally, it is relatively inflexibly and lack biomolecular signals, causing compliance mismatch, lack of functional endothelium and lack of self-healing capability. It is well known that hemodynamic flow disturbance caused by compliance mismatch between vascular graft and native blood vessels contributes to the failure of vascular grafts.

Therefore, there is a need for ePTFE replacement materials for new A/V graft products with characteristics allowing early implantation and long-term patency (i.e., a low long-term failure rate) on hemodialysis patients.

### SUMMARY OF THE INVENTION

Some aspects of the invention provide bionanocomposite materials comprising at least two coaxial layers of bionanocomposites and having an interior that is capable of allowing a fluid to flow therethrough. Such bionanocomposite materials typically comprise:

a core structure; and

a shell structure encapsulating the core structure.

One of the core structure or the shell structure comprises a biocompatible natural-derived material and the other comprises a biocompatible biomimetic nanostructure. Moreover, there can be additional layers of materials. Such additional layers can be present in the interior of the core structure, on the exterior of the shell structure, inbetween the core structure and the shell structure, or a combination thereof.

Typically, the bionanocomposite materials of the invention have inner diameter for about 10 mm or less, often about 8 mm or less, more often about 6 mm or less, and most often about 4 mm or less. In other embodiments, the inner diameter of bionanocomposite materials of the invention is at least about 2 mm, typically at least about 4 mm, and often at least about 6 mm. Such bionanocomposite materials can be used in a variety of applications including, but not limited to, medical conditions including, but not limited to, coronary artery grafts, bypass grafts (e.g., for atherosclerosis), pediatric shunt grafts (e.g., for congenital heart diseases), arteriovenous (A/V) grafts (e.g., for vascular access in hemodialysis patients with kidney failure), treatment for aneurism (AAA and TAA), as well as other clinical conditions that can benefit from artificial grafts. It should be appreciated, however, that the scope of the invention is not limited to such uses. In fact, bionanocomposite materials of the invention can be used in non-medical applications such as material sciences, medical devices, etc. It should be appreciated that the outer diameter of bionanocomposite materials of the invention is not limited to those stated above. In fact, depending on a particular application and/or the need, the outer diameter of bionanocomposite materials of the invention can vary widely.

In some embodiments, the bionanocomposite material is tube-like or in a tubular form. As used herein, tube-like or a tubular form refers to a substantially circular, elliptical, or oval like shape and having an interior that is either porous or hollow such that a fluid medium can flow therethrough.

Yet in other embodiments, the core structure comprises a porous biocompatible natural-derived material and the shell structure comprises a biocompatible biomimetic nanostructure. The term “natural-derived material” refers to material that is not synthesized by human or a material that is biologically-derived. While such a material can be isolated and/or purified from a natural source, it is not chemically or artificially synthesized. The term “biomimetic” refers to human-made processes, substances, devices, or systems that imitate or act similar to nature. In other embodiments, the shell structure comprises a porous biocompatible natural-derived material and the core structure comprises a biocompatible biomimetic nanostructure.

Still in other embodiments, the biocompatible natural-derived material is biodegradable. Within these embodiments, in some instances the biocompatible natural-derived material comprises a biodegradable natural-derived composite hydrogel. Within these instances, in some particular cases, the biodegradable natural-derived composite hydrogel comprises collagen, chitosan, elastin, or a combination thereof.

In other embodiments, the core structure further comprises vascular smooth muscle cell, a bioactive material, or a combination thereof.

In some particular embodiments, the core structure is capable of facilitating cell adhesion, in-growth, remodeling, or a combination thereof.

Yet in other embodiments, the biocompatible biomimetic nanostructure comprises fibroin, collagen, a biodegradable polymer, or a combination thereof or a derivative thereof. Still in other embodiments, the shell structure comprises fibroin, collagen, polycaprolactone, or a combination thereof.

Still in other embodiments, the shell structure further comprises a signaling compound that is capable of providing long-term remodeling signal in vivo.

In some particular embodiments, the bionanocomposite material has an average burst strength ranging from about 1200 mmHg to about 2400 mmHg. In other embodiments, the bionanocomposite material has an average modulus of from about 3 MPa to about 15 MPa.

Typically, the bionanocomposite materials of the invention allow a fluid flow within the inner diameter (e.g., axially) but prevents any significant liquid permeability through the walls of the material.

The core structure can also comprise a core additive substance that promotes endothelial cell adhesion or regulates cell proliferation, or both. In one particular embodiment, the core additive substance comprises heparin.

The shell structure can also comprise a shell additive substance that promotes smooth muscle cell differentiation, self-healing process, or a combination thereof. In some embodiments, the shell additive substance comprises TGF-β.

In some embodiments, bionanocomposite materials are multilayer structure. In some instances such materials form intima-equivalent and/or media-equivalent layers.

Other aspects of the invention provide methods for producing the biocompatible biomimetic nanostructure disclosed herein. One particular embodiment provides a method for producing a tubular bionanocomposite material comprising at least two coaxial layers of bionanocomposites. Such a method typically comprises:
placing a core structure comprising a porous biocompatible natural-derived material on a mandrel; and
electrospinning at least two different biocompatible shell structure materials on to the core structure to form a shell structure comprising a biocompatible biomimetic nanostructure that encapsulates the core structure, whereby a tubular bionanocomposite material comprising at least two coaxial layers of bionanocomposites with a porous core structure is produced.

Some embodiments of methods of the invention allow gradient distribution of fibers and/or impregnated molecules. Such embodiments allow achievement of spatiotemporal control over the biomolecules on the luminal side and/or the abluminal side.

In some embodiments, the step of electrospinning at least two different biocompatible shell structure materials is simultaneously applied to the moving core structure on the mandrel to produce a substantially intertwined shell nanostructure.

Still in other embodiments, the core structure is produced by a self-assembly process. In some particular embodiments, the core structure comprises self-assembled composite gel. Exemplary composite gels include, but are not limited to, collagen, chitosan, elastin, and a combination thereof.

Yet in other embodiments, the biocompatible shell structure materials comprises fibroin, collagen, a biodegradable polymer, or a combination thereof or a derivative thereof. Still in other embodiments, the biocompatible shell structure materials comprises fibroin, collagen, polycaprolactone, or a combination thereof.

Still in other embodiments of methods of the invention, the core structure further comprises vascular smooth muscle cell, a bioactive material, or a combination thereof. In one particular embodiment, the bioactive material comprises heparin.

In other embodiments of methods of the invention, the shell structure further comprises a bioactive material. Within these embodiments, in some instance the bioactive material comprises a signaling compound that is capable of providing long-term remodeling signal in vivo.

In some embodiments, the bioactive material is added to a solution of the biocompatible shell structure material prior to the step of double-electrospinning.

Yet still in other embodiments, the bioactive material comprises TGF-β.

Still other aspects of the invention provide tubular bionanocomposite materials comprising a core structure and a shell structure. Typically, the core structure comprises a porous fiber structure, while the shell structure generally comprises a tubular layer of strong, highly-dense electrospun nanofiber.

In some embodiments, the core structure comprises a material that is typically self-assembled or electro spun (often comprising two or more different materials). Generally, the core structure comprises biodegradable materials such as fibroin, PCL, PDLLA, PLA, PGA, PGLA, collagen, chitosan, elastin, etc., or a combination thereof. Typically, the core structure comprises PCL, PDLLA, fibroin, or a combination thereof. The core structure can also include an additive substance such as an anti-coagulant (e.g. heparin, antithrombin, etc), a growth factor that stimulates endothelial function, or a combination thereof.

In other embodiments, the shell structure comprises a porous synthetic or biological material, or a combination thereof. Typically, the shell structure comprises a material such as PCL, PDLLA, PLA, PGA, PGLA, collagen, chitosan, elastin, fibrin, hyaluronic acid, fibroin, or a combination thereof. The shell structure can also include a bioactive additive substance, a growth factor such as PDGF, FGF, TGF-B for smooth muscle cell in-growth, or a combination thereof.

Still in other embodiments, compositions of the invention can optionally include cells such as endothelial cells, smooth muscle cells, or a combination thereof.

Yet in other embodiments, the tubular bionanocomposite materials of the invention have an average burst strength ranging from about 1200 mmHg to about 2400 mmHg. In other embodiments, the tubular bionanocomposite materials of the invention have an average modulus of from about 100 KPa to about 10 MPa, typically from about 500 KPa to about 5 MPa, and often from about 1000 KPa to about 1 MPa.

### BRIEF DESCRIPTION OF THE DRAWINGS

FIG. 1 is a schematic illustration of one embodiment of a multitubular biograft construct of the present invention.

FIG. 2A is a graph showing arteries complying with J-shaped curve.

FIG. 2B is a graph showing variation of compliance v. mean pressure for different conduits. Porous compliant poly(carbonate)-polyurethane (CPU) is a compliant prosthesis.

FIG. 3A is a graph showing a characteristic stress-strain curve for collagen (COL), collagen-chitosan (COL-CHIT), and collagen-chitosan-elastin (COL-CHIT-ELN).

FIGS. 3B-3D are bar graphs of tensile testing results for matrices cross-linked with different cross linkers: *:p<0.05 wrt EDAC †:p<0.05 wrt FLD **:p<0.1 wrt EDAC ††:p<0.1 wrt FLD.

FIG. 4 is a graph showing anisotropic characteristics of materials produced from electro spinning with rotational mandrel.

FIG. 5A is SEM image of electrospun fibroin net and smooth muscle cell.

FIG. 5B is SEM image of electrospun fibroin net of FIG. 5A after 4 days of culture.

FIG. 6A is a bar graph showing the effects of relative humidity (R.H.) and spinning voltage on the average diameter of electrospun nanofiber.

FIG. 6B is a bar graph showing the effects of fibroin concentration on the average diameter of electrospun nanofiber.

FIG. 7A is a schematic of double-electrospinning system.

FIG. 7B is a FTIR showing the existence of two intertwined fibers.

FIG. 8 is a schematic representation of nanostructures that facilitates controlled release of bioactive molecules in the nanocomposites of the constructs: heparin/chitosan particles and TGF-β in the gradient of silk-fibroin electrospun nanofiber.

FIGS. 9A-9E shows: (A) Illustration of the interpenetrating nanofiber structure from double-electrospinning; (B) FTIR of various nanofiber compositions; (C) Compositional gradient; (D) High mechanical elasticity in both circumferential and longitudinal direction of nanofiber-based tubing; it varies with mandrel rotation speed during synthesis; (E) Degradation and hydration of homogeneous nanofibers and interpenetrating nanofibers shown by DSC.

FIG. 10 is a schematic illustration of a structure of a novel core-shell bilayer graft: the core comprises interpenetrating degradable nanofibers for endothelium formation; the shell comprises porous collagen-chitosan scaffold for formation of vascular media-equivalent. Biomolecule signals such as PDGF and heparin are spatially-defined as cell-specific microenvironments. The concentration gradient and controlled release of heparin is facilitated by nanofibers.

FIG. 11 shows proper interface design needed to lead to increasing the adhesion between the two layers.

FIG. 12 is SEM images of molecule-impregnated nanofibrous degradable composites that are suitable for vascular grafting.

FIG. 13 is a bar graph of biological characterizations showing the SMC contraction of the collagen-chitosan scaffold.

FIG. 14 is images showing healing of a needle puncture hole (mimicking vascular access) in the scaffold.

FIGS. 15A-15C are graphs of mechanical characterizations of the porous scaffold showing failure stress, % of elongation, and elastic modulus, respectively.

FIG. 16 is a bar graph showing ingrowth depth of SMC in the collagen-chitosan scaffold.

FIG. 17A is a schematic illustration of PCL double electrospun with PEO and then with an aqueous solution of PEO.

FIG. 17B is a schematic illustration showing top layer of PCL that is spray-coated or double electrospun with Collagen.

### DETAILED DESCRIPTION OF THE INVENTION

Hemodialysis is used to filter blood when kidneys can no longer do so. A vascular access is needed to circulate the blood for hemodialysis because the normal blood vessels cannot handle the high blood flow speeds required for this procedure. These grafts often develop hyperplasia, a condition in which vascular cells overgrow onto the artificial tubing and cause stenosis or narrowing vascular lumen, and even complete blockage. The blockage in the graft can impede blood flow and make hemodialysis difficult. Hyperplasia can further cause other cardiovascular complications in the patients. Hyperplasia occurs frequently, causing the failure of 40 to 50% of hemodialysis grafts in the first year. Failure of AV grafts is also associated with thrombosis. It is believed that both hyperplasia and thrombosis result from several pathogenic factors including, but not limited to: (1) hemodynamic flow disturbance caused by compliance mismatch between A/V graft and native blood vessels; (2) lack of functional endothelium and molecule signals on the graft to control cell proliferation; (3) susceptibility of inflammation and infection at the dialysis needle puncture sites due to lack of self-healing; and (4) a combination thereof. All these pathogenic factors are associated with mechanical and biological properties of the current graft standard, expanded polytetrafluorylene (ePTFE), a synthetic material that is stiff, inert (i.e., low cell-adhesive), and biological-signal-deficient (e.g., signals for maintaining proper smooth muscle function). Despite the magnitude of graft-associated problems, there are currently very few effective therapies that address the problems of vascular access dysfunction.

Though various approaches have been used to develop alternative materials to ePTFE for small-diameter vascular grafts primarily for coronary artery bypass, they are yet to demonstrate success in vivo and clinically. Some of the current approaches, including synthetic biomaterial engineering, xenograft processing and tissue engineering, have developed a range of new potential graft materials that demonstrate improved compliance or surface properties for endothelial adhesion. As the most recent advance, tissue engineering uses a combination of cells and scaffolding materials to develop viable tissue-like vascular constructs by in vitro culture or in vivo techniques (with in vitro culture as the most widely-adopted strategy). Often, tissue engineering strategies are aimed at properly replicating a structural and functional component of vascular layer (e.g., media layer) to coax physiological functioning and remodeling of vascular cells.

Native arteries are structured into what is generally considered to be 3 layers: intima, media and adventitia. Each layer is unique in cell, molecule and extracellular matrix (ECM) compositions to fulfill a specific physiological function. For example, intima contains endothelial cell (EC) for inhibiting thrombosis and regulating SMC function, and media contains smooth muscle cell (SMC), collagen and elastin for gaining contraction, elasticity and healing. Tissue engineering, producing products that resemble native arteries, represents a highly promising approach for the future of vascular surgery. However, a number of obstacles still must be overcome before this enormous engineering feat can be realized. Current vascular tissue-engineering approaches lack spatially and temporally precise cues (including structural and molecular signals) in the constructs to robustly guide the maturation of vascular structure and functionality. More importantly, tissue-engineered constructs are yet to address several clinical-related concerns including time, cost and efforts involved, possibility of contamination, and quality control over the long course of in vitro production.

Some aspects of the invention advances vascular tissue engineering by providing compositions and methods for producing novel biomimetic constructs. In some embodiments, compositions of the invention comprise multi-layer structure, ECM-like degradable nanofibers, and/or spatially-defined and temporally-defined signaling molecules. In some instances, the release of cell-specific biomolecules are spatially defined in each layer and temporally controlled via the nanofibrous structure, in order to simultaneously achieve multi-functionality (e.g., anti-thrombosis, anti-hyperplasia and pro-healing). In many instances, the structure and composition are tailored to achieve desired properties to induce favorable vascular remodeling and long-term integration in vivo. In some particular embodiments, the design and fabrication of the novel graft device are aimed at easy packaging and transport, and clinically “off-shelf” availability.

Small-diameter (i.e., ≦6 mm) ePTFE vascular grafts represent the limitation of modern biotechnology. In addition to A/V vascular access graft for hemodialysis patient, ePTFE vascular grafts are used for a variety of medical conditions encompassing coronary artery bypass grafts for atherosclerosis and pediatric shunt grafts for congenital heart diseases. In these applications, graft failure also accounts for numerous complications and mortality.

Some aspects of the invention provide bionanocomposite materials and method for producing and using the same. In some embodiments, bionanocomposite materials of the invention can be used as vascular graft materials that eliminate or significantly reduce the problems associated with ePTFE vascular grafts. In some embodiments, vascular graft materials of the invention have arterial-like mechanical and biological properties and can be personalized and assembled in a clinical laboratory setting within a few days, typically less than 7 days, often less than 4 days, and more often within 1-2 days.

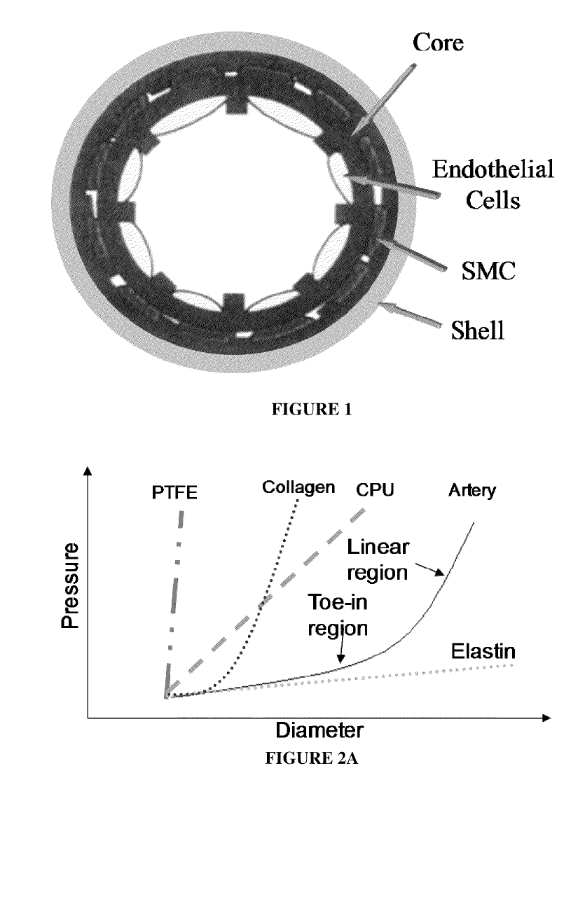

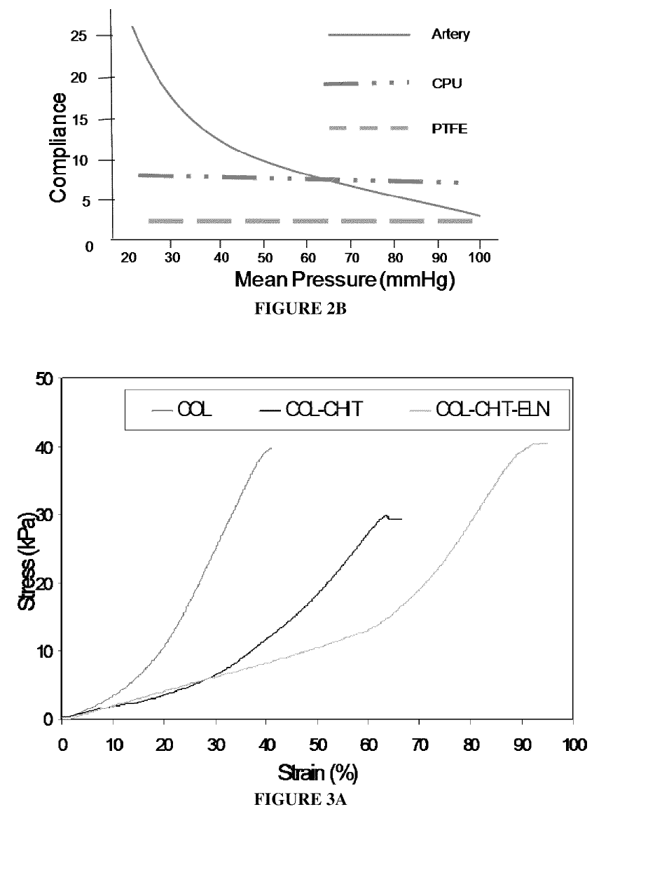

In other aspects of the invention, bionanocomposite materials of the invention comprise soluble, biodegradable, natural-derived materials (e.g., collagen, chitosan, elastin and silk-fibroin) and biomimetic nanostructure (e.g., nanofiber) fabricated by a bionanocomposite material fabrication technique combining self-assembly, double-electrospinning and tissue engineering approaches. Some embodiments within these aspects of the invention are based on the coaxial multiple layer tubular graft that is assembled by two different types of bionanocomposite tubes: the inner tube (the core) is made of biodegradable natural-derived composite hydrogel (e.g., collagen-chitosan-elastin) compacted with, for example, vascular smooth muscle cells; the outer tube (the shell) is made of fibroin or its composite with collagen-based materials. An exemplary configuration of this particular embodiment is shown in FIG. 1, which shows multi-tubular biograft construct. The core is higher in compliance (or elasticity) and weaker in strength thus contributing to most of the elastic “toe-in” region at the low pressure range of the pressure-diameter curve, while the shell is typically lower in compliance and higher in strength (relative to the core) thus contributing to the stiff linear region at the high pressure range of the curve. By increasing the intraluminal hydrodynamic pressure, the core can markedly inflate in the low-pressure regions, and after the core comes into contact with the outer tube, both tubes inflate together gradually in the high pressure regions. The stress-strain relation or mechanical behaviors of this graft generally follow the J-shaped curve of arteries as shown in FIG. 2. The figure also shows that the strain of synthetic materials respond to stress change with a substantially linear relationship. To minimize flow disturbance, a closely matched compliance of graft material is generally used. Desired mechanical properties (e.g., strength, compliance and compliance-pressure relationship) for vascular grafts can be achieved with the compositions of the invention.

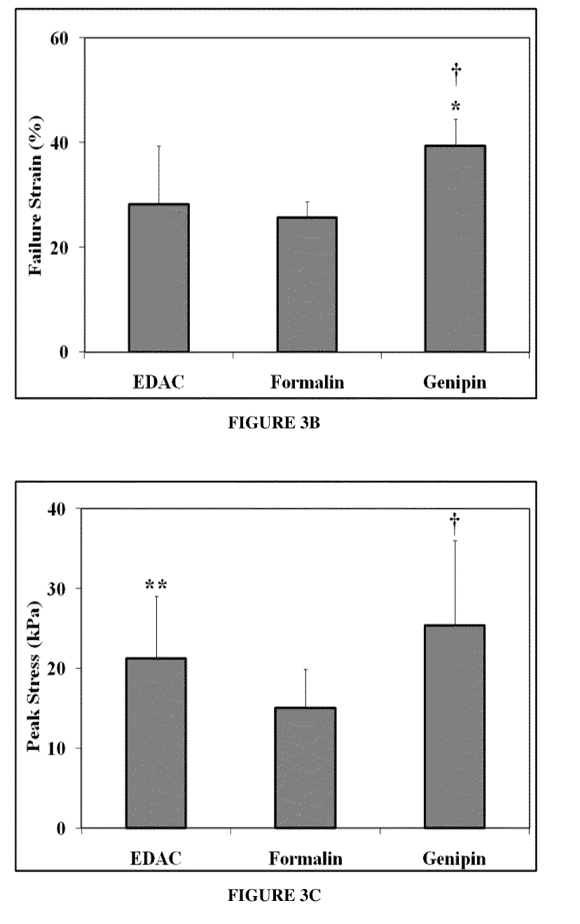

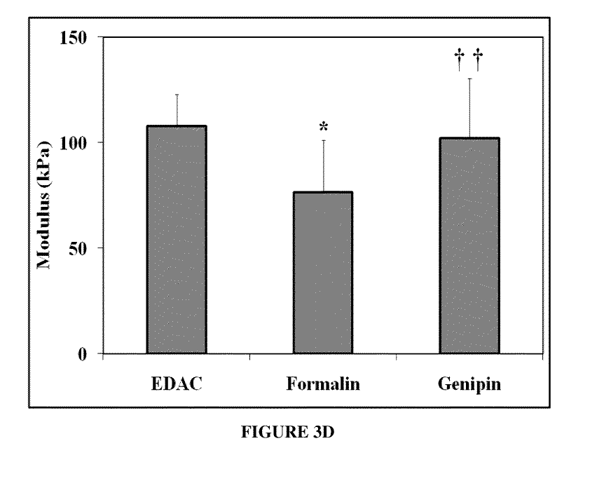

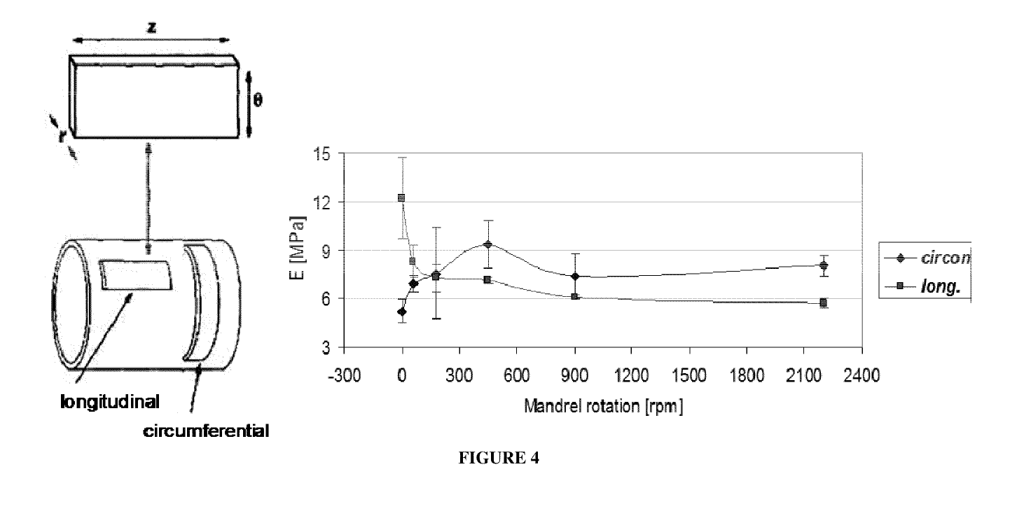

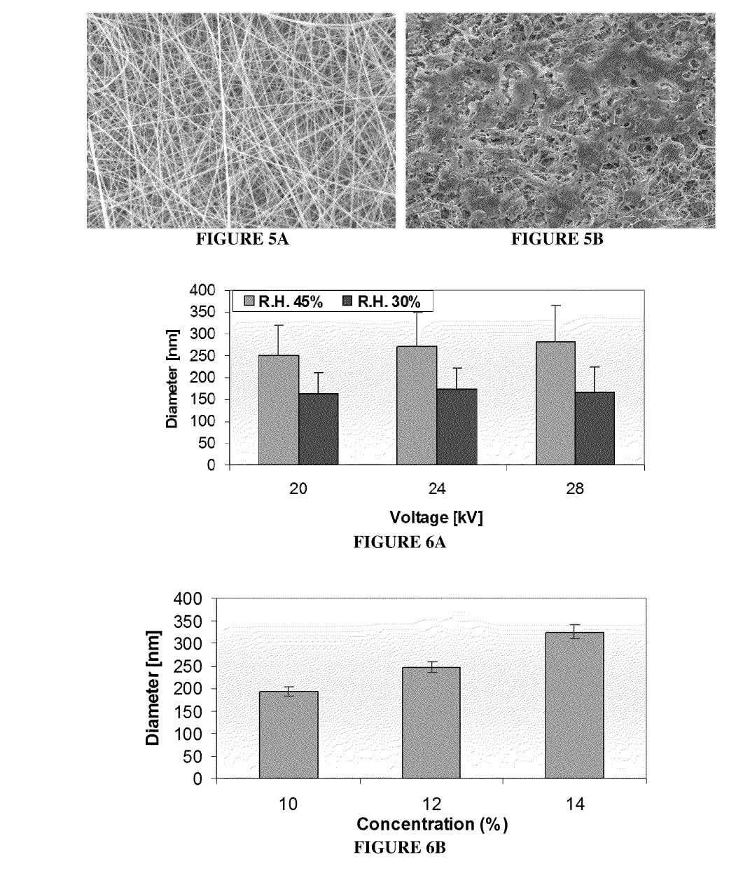

Some tubular grafts of the invention have coaxial multiple layer with a cell-compacted, self-assembled, elastic core and a relatively strong shell that is produced by double-electrospinning, co-electrospraying, or a combination thereof. In some embodiments, the core material of the invention showed similar shaped curve of stress-strain relationship and the strength reached 30 Kpa after cross-linking (FIGS. 3A-3D), while the shell showed anisotropic characteristics of tubular structure with higher strength and modulus (FIG. 4). Also, both materials demonstrate biomimetic nanofiber structure. See FIGS. 5A and 5B. In other embodiments, cell seeding in multilayer configuration with the materials provide enhanced biological compatibility. Accordingly, some aspects of the invention provide compositions and method for producing bionanocomposite materials having biomimetic mechanical behaviors. Some methods of the invention include using nanomaterial processing techniques which can be tailored to substantially replicate biomimetic mechanical properties of a native vessel. In particular, some vascular graft materials of the invention have a high strength and artery-like compliance.

Still other aspects of the invention provide multilayer bionanocomposite materials that can be used in a variety of biomedical applications including, but not limited to, construction of early and long-term vascular access for hemodialysis patients. In some embodiments, the bionanocomposite materials have multilayer configuration, novel composition, and/or nanostructure.

In some aspects of the invention, the bionanocomposite materials are characterized with a multi-tubular structure with an inner tube (i.e., core) comprising a natural-derived material (e.g., collagen-chitosan nanocomposite) and an outer tube (i.e., shell) comprising a nanocomposite material (e.g., fibroin-PCL nanocomposite). Without being bound by any theory, it is believed that the shell provides high strength, ensures low permeability and long-term remodeling signals, while the core provides blood-compatible and cell-adhesive surface for easy implantation. In some embodiments, all the materials are biocompatible and biodegradable. As used herein, “biocompatible” means being biologically compatible by not producing a significant toxicity, injury, or immunological response in living tissue. And the term “biodegradable polymer” refers to a synthetic polymer that is degraded to a smaller fragment by a biological system. Biodegradable polymers typically have half-life of about a year or less, often about six-months or less, and more often about a month or less. It is also believed that when the bionanocomposite materials are grafted into a subject, cells in vivo replaces graft material over time, and form natural vessels, thereby eliminating a need for graft replacement. The multilayer bionanocomposite materials offer the potential for realizing an easy, long-term grafting platform.

Some bionanocomposite materials of the invention exhibit designs and functions that mimic natural arteries and/or avoid or significantly reduce pathogenic cellular mechanisms leading to graft failure of other conventional materials. Bionanocomposite materials of the invention include one or more of the following characteristics/properties: (i) mechanically strong and having cell-compatible and blood-compatible surface that allow early implantation; (ii) matching compliance that avoid flow disturbance at the junction of graft and native vessels; (iii) a functional endothelium coating due to the abundance of RGD sequence on the core (e.g., collagen-chitosan material); (iv) capability of self-healing due to biomolecular signals in the shell inducing the migration of vascular smooth muscle and fibroblast cells; (v) reducing or eliminating occurrence of thrombosis, stenosis and/or intima hyperplasia due to minimal flow disturbance, confluent functional endothelium and self-healing capability; and (vi) controlled biomolecular signals to direct remodeling and degradation in vivo.

Additional objects, advantages, and novel features of this invention will become apparent to those skilled in the art upon examination of the following examples thereof, which are not intended to be limiting. Throughout this disclosure, procedures that are constructively reduced to practice are described in the present tense, and procedures that have been carried out in the laboratory are set forth in the past tense.

### EXAMPLES

The present inventor has developed a set of bioengineering techniques, including electrospinning (typically double-electrospinning) synthesis techniques for degradable biohybrid nanofiber composites with 3-dimensional signal gradients, tissue engineering techniques for vascular media-equivalent scaffolds that are mechanically strong and biological functional, as well as advanced imaging techniques for non-invasively monitoring the dynamics and interactions of cell, molecule and matrix in engineered tissues. In addition, methods have been developed for investigating molecular and cellular mechanisms underlying inflammatory response by vascular endothelium under biomimetic pulsatile flow conditions as well as mechanisms underlying the interactions between vascular endothelium and vascular smooth muscle. A porcine model has been developed to study pathology of ePTFE A/V grafts, including assessing neointimal hyperplasia, thrombosis, short-term and long-term biological evaluation of hemodialysis A/V grafts in the porcine model.

### Material Design and Functionality for Tubular Vascular Grafts

The present inventors have discovered methods and apparatuses for fabricating and characterizing bionanocomposite materials that can be used in a variety of applications including, but not limited to, biomaterials and biomimetic structures for blood vessels. Disclosed herein are some aspects of methods and devices for fabricating or producing various bionanocomposite materials. Also disclosed herein are some of the mechanical and biological properties of exemplary bionanocomposite materials such as, but not limited to, collagen-chitosan-based nanocomposite, and fibroin-polycaprolactone (PCL) nanocomposite. Biological and mechanical properties of bionanocomposite materials of the invention can be modified by a variety of factors including, but not limited to, controlling fabrication techniques, materials used in production of bionanocomposite materials, as well as integrating mechanical and biological behaviors of the compositions of the bionanocomposite materials, for example, by introducing a multilayer configuration to combine the two composites by forming an inner tube (i.e., core) and an outer tube (i.e., shell). By using the fabrication technique and quantitative determination of fabrication parameters disclosed herein, one skilled in the art having read the present disclosure can produce or fabricate the bionanocomposite materials having desired mechanical and biological properties. Such bionanocomposite materials can be used to construct multi-layer materials that can be used in a variety of applications including, but not limited to, A/V grafts.

### Design and Fabrication

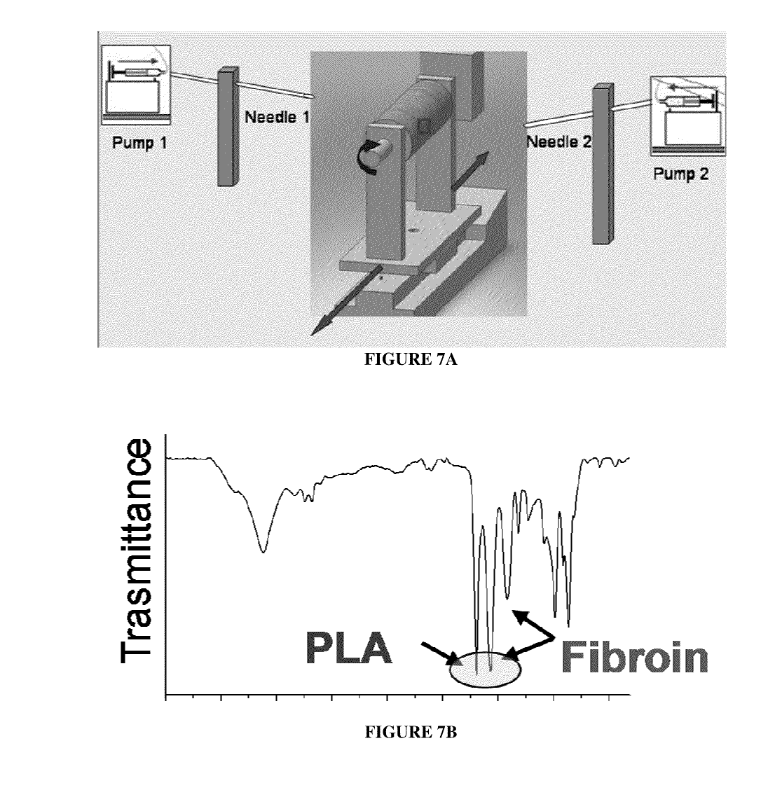

One embodiment of the design and fabrication of the bionanocomposite materials is shown in FIG. 7A. In FIG. 7A, the tubular graft of bionanocomposite material is constructed from two coaxial layers of bionanomaterials: the core composed of self-assembled collagen-chitosan composites and the shell composed of fibroin-PCL composites. See FIG. 7B. Collagen and chitosan are both naturally-derived materials that contain molecular sequences that facilitate cell adhesion, in-growth and remodeling. Collagen-chitosan composites form 3-dimensional porous coating on the lumen and can induce seeding of functional endothelial cell (EC) (e.g., to form a non-thrombogenic surface) and in-growth of smooth muscle cell (SMC). Fibroin is a slowly-degradable biological material isolated from silk while PCL is a highly-biocompatible, slowly-biodegradable synthetic material. The fibroin-PCL nanofiber shell can form a strong and highly-dense wrap around porous gel core to provide desired mechanical properties at the time of implantation. In addition, methods and compositions of the multilayer bionanocomposite of some aspects of the invention also include layer-specific biomolecules immobilized in the constructs. Biomolecules such as heparin which can attract EC and regulate cell proliferation are immobilized by forming complexes with chitosan in the core; while biomolecules such as TGF-β which can promote SMC differentiation and self-healing process are embedded in fibroin nanofibers in the shell. Thus, the release of cell specific molecules can be spatially defined in each layer.

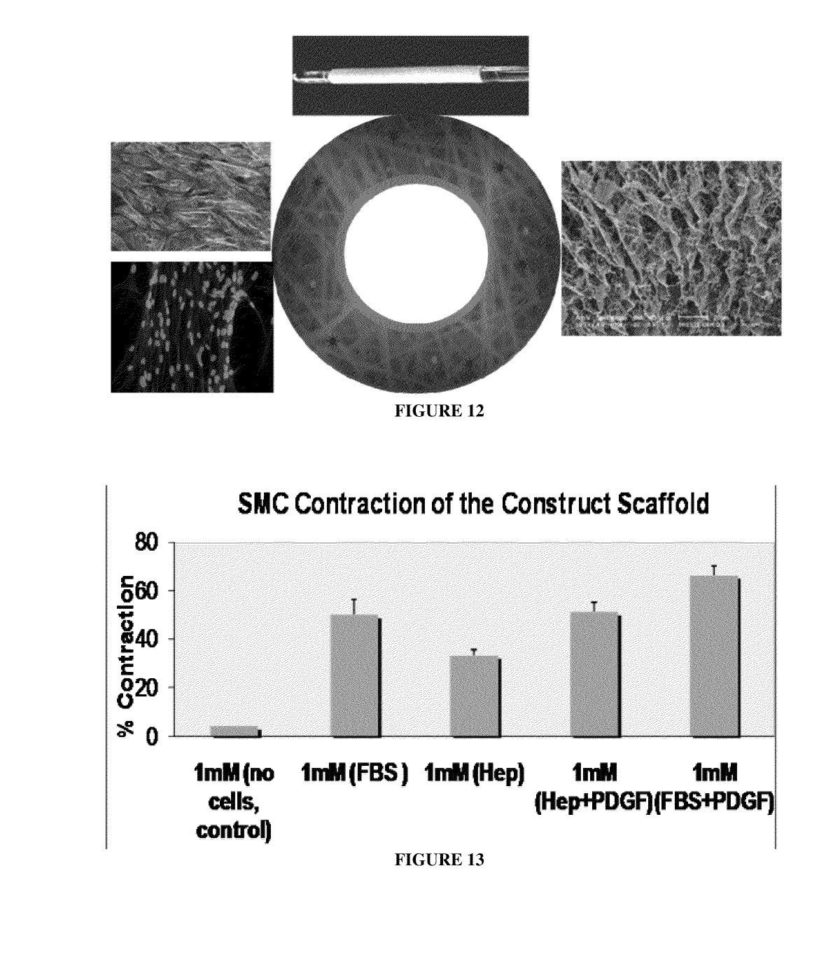

In some embodiments, the fabrication process comprises (1) developing self-assembled collagen-chitosan composite gel; and (2) double-electrospinning fibroin and PCL nanofibers on a rotational mandrel coated with collagen-chitosan gel. The incorporation of heparin in collagen-chitosan gels employs a joint precipitation process of cationic chitosan and anionic heparin in the form of polymeric nanoclusters caused by the addition of a cross-linking agent such as sodium tripolyphosphate. In some embodiments, a double-electrospinning process developed by the present inventors is used to prepare a gradient distribution of TGF-β through the gradient blend of nanofibers in the composites. See, for example, FIG. 12. In some instances, TGF-β molecules are added into the fibroin solution before electrospinning.

### Material Fabrication Techniques Based on the Mechanical Properties

Because some aspects of the mechanical properties of the bionanomaterial composites of the invention are determined by the shell, in some embodiments the mechanical properties (e.g., strength and elasticity) of bionanomaterial composites can be modulated by modulating the spinning process for individual component and/or the moving speed of the mandrel to achieve the desired properties. Through double-electrospinning, the nanostructure and the density of fibroin nanofibers and PCL nanofibers can be separately controlled (for example, with voltage, relative humidity and/or polymer solution concentration, see FIGS. 6A and 6B). In some embodiments, double-electrospinning is simultaneously applied to a moving mandrel jacketed with composite gel to produce a substantially intertwined shell nanostructure. It should be appreciated that depending on the fabrication process, the intertwined shell nanostructure can be formed in only a desired portion or all of the electrospun mesh with nanofiber gradients over the thickness. The gradients of the nanofibers can be controlled by, for example, varying comparative flow rates and moving speed of the collection mandrel.

Strip samples from the circumferential direction and the longitudinal direction of tubular constructs can be taken and measurements can be performed to determine various mechanical and physical properties. For example, samples can be placed into an Insight tensile electromechanical testing system with an environmental bath of 1×DPBS (Instron 4502; Instron, Norwood, Mass.) to measure, for example, dimensions such as thickness, gage length and width. The samples can be clipped onto the grips which are connected to, e.g., a 5 N load cell and tested at a strain rate of 1% per second. Stress-strain diagrams can be rendered with the aid of a computer (Software Series IX, Instron). Samples are typically tested until the breaking point.

### Bioactive Molecules in the Constructs Using In Vitro Cellular Evaluation Experiments

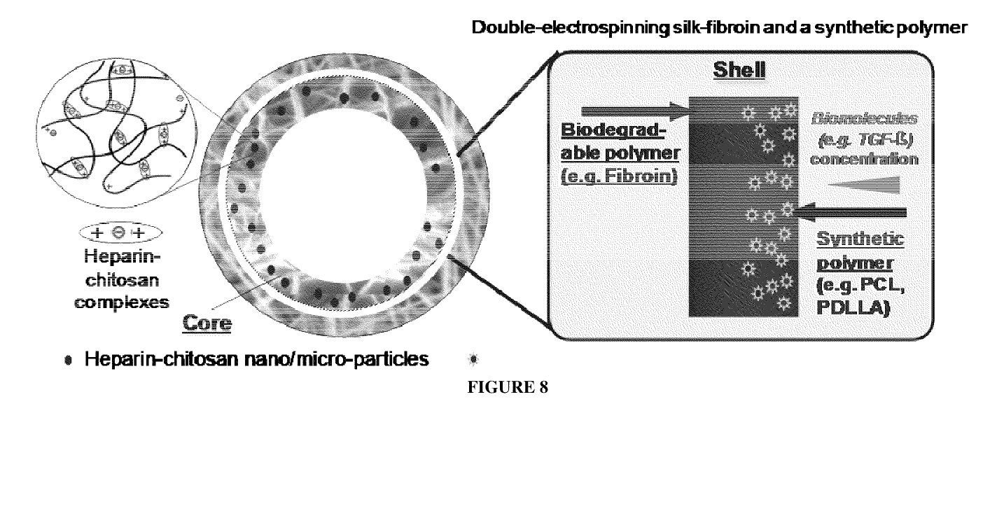

Multilayer nanocomposite samples without bioactive molecules (e.g., drugs) and multilayer nanocomposite samples with bioactive molecules loaded at various concentrations are produced. See, for example, FIG. 8. In vitro cell experiments are carried out to determine the effective bioactive molecule concentration. To evaluate how the release of bioactive molecules, e.g., heparin or TGF-β, influences cell behaviors, experiments were performed to determine EC function seeded on the constructs and to examine SMC differentiation and proliferation. The samples without bioactive molecules were used as controls. To demonstrate molecular effects on the endothelium, the constructs with or without bioactive molecules were seeded with endothelial cells and cultured for 4 weeks. Samples are taken weekly and endothelial function molecules such as eNOS, PGI2, VEGF and PDGF are analyzed with Western blot. Similar culture conditions are applied to SMCs and SM α-actin, SM MHC and PCNA are analyzed with Western blot and immunofluorescence.

### Physical and Mechanical Tests on the Graft Device

To perform in vitro functional testing, the tubular graft constructs are produced with desired physical and mechanical properties as described herein. The size of the tube is ID 4 mm and OD 5.5 mm. Twenty (20) samples are tested for the following properties for functional evaluation of vascular access: (1) longitudinal tensile strength; (2) circumferential burst strength; (3) burst strength after repeated puncture; (4) suture retention strength; (5) dynamic compliance; (6) water permeability; (7) wall thickness; (8) visual inspection; (9) usable length; (10) pressurized internal diameter; and (11) relaxed internal diameter. Suture retention and burst strength in different directions test durability and integrity of the graft device. Permeability and compliance are tested in a circulation model.

The results of the in vitro testing demonstrate the safety and effectiveness of bionanocomposite materials of the invention. In some embodiments, bionanocomposite materials of the invention achieve a sufficiently low level of permeability (<5 mL/cm²/min at 120 mm Hg), and achieve sufficiently high levels of burst strength (F>200 N/cm) in different testing orientations as well as sufficiently high levels of suture retention strength (F>30 N/cm) in different testing orientations. The level of permeability can be modulated by modulating the density of the nanofibers, e.g., by adjusting the electrospinning rate. The strength of bionanocomposite materials of the invention can be modulated by, e.g., modulating the spinning parameters in the corresponding direction.

### In Vivo Tests on the Graft Device

To demonstrate in vivo biocompatibility, functionality and advantage over ePTFE, bionanocomposite materials of the invention are tested on an animal model. In selecting the appropriate animal model, efforts are made to evaluate device performance in models that demonstrate performance attributes in a clinically meaningful fashion. The porcine model is used to assess the following one or more aspects of the bionanocomposite material graft to: (1) provide early vascular access for hemodialysis; (2) determine the biological response to the implanted grafts; (3) be able to remodel and form into arterial-like structure; and (4) provide long-term patency with minimal intima hyperplasia, stenosis and thrombosis. Using a porcine to study pathology of ePTFE A/V grafts are well known to one skilled in the art. A similar experimental procedure is used to study pathology of bionanocomposite material grafts of the invention.

The bionanocomposite material grafts of the invention are properly grafted with minimal difficulty or complications. No twisting or kinking of the device are observed after implantation. The majority of the devices remain patent during 180-day period. Stenosis of the device over time is minimal. There is a minimal inflammation response caused by a device implant. Limited adverse biological reactions to the bionanocomposite material graft is observed compared to ePTFE grafts.

The strength of the enabling technology proposed here is that it addresses most of the known pathogenic problems associated with long-term failure of ePTFE grafts. And it facilitates fast maturation for easy grafting. The results from proposed studies will help to evaluate the potential success of this product. If successful, the product can be used, for instance, for haemodialysis patients who require easy and routine access to the bloodstream, a prosthetic blood vessel can be used within one week after implant to provide entry and exit points for needles carrying blood to and from haemodialysis equipment. However, as ePTFE dominates the current market occupying more than 90% of the market of vascular graft market for more than 3 decades, it may take time for the vascular surgeons to switch to a new product. Also, the initial cost of the proposed product might be a little higher. Other more recent approaches to small-diameter vascular grafts, including synthetic biomaterial engineering and tissue engineering, have developed a range of new graft materials that demonstrate improved compliance or modified surface to enhance endothelial adhesion. In particular, tissue engineering, integrating cells and biomaterials into engineered constructs showing tissue-like functions, represents a highly promising approach to current vascular grafting. However, a number of obstacles still must be overcome before this enormous engineering feat can be realized. Tissue-engineering approaches are challenged to deliver spatially and temporally precise signals in vascular constructs to guide construct maturation, function and integration with native blood vessels. More importantly, tissue-engineered constructs are yet to address several clinical related concerns which are all relevant to the long course of in vitro tissue production. These include time, cost and efforts involved, possibility of contamination, and quality control. Therefore, the proposed approach here can advance vascular tissue engineering by implementing new strategies that involve the design of novel biomimetic constructs with nanoscale features tailored to achieve precise control over biological signals to direct tissue growth.

### Electrospinning Process

One particular embodiment of the electrospinning process and system is shown in FIG. 7A. This example exemplifies a double-electrospinning process for producing nanocomposites, e.g., made from natural-derived silk-fibroin and synthetic degradable materials such as PCL and/or PDLLA. The present inventor also developed a double electrospinning technique for producing a tubular structure of heterogeneous interpenetrating nanofibers with a gradient blend of the fibers.

Briefly, the composite materials were fabricated with two simultaneous feeding syringes: one with silk-fibroin in formic acid solution and the other with PCL in chloroform solution. The nanofiber diameters of fibroin and PCL were separately controlled with different electrospinning systems. Pure fibroin powder was regenerated and dissolved in neat formic acid at various concentrations. The positive electrode of a high-voltage power supply was directly connected to the needles to charge the spinning drop of polymer solution, and the negative electrode was clipped in the collector. The syringes were placed on syringe pumps with the capability of regulating flow rate and maintained a constant flow. The electric potential, the solution flow rate, and the distance between the needle tip and the aluminum foil were adjusted until a stable jet was obtained. The entire apparatus was mounted in a sterile hood and nitrogen gas was fed continuously around the system during the electrospinning process to control the humidity which was regulated to a range of 20-30%. The syringe flow rate can be controlled manually or with computer control system.

After preparation, all samples were dried under vacuum prior to the analysis. For strip testing, a routine electrospinning setting with a flat collecting plate was used and the final material was cut into a dog-bone shape. For a tubular structure, the electrospinning system was combined with a rotational mandrel that can be moved in the lateral and transversal directions. This facilitated the fabrication of gradient blend of silk-fibroin nanofibers and PCL nanofibers. The electrospinning parameters (e.g., voltage, feeding rate, and/or relative humidity) were controlled to achieve desired nanostructural properties such as fiber diameter and porosity. The movement of the mandrel was controlled to achieve the nanofiber gradients and the circumferential/longitudinal modulus ratio. The anisotropic modulus ratio was demonstrated as discussed herein.

### Measurement of Stress-Strain Curves

Tensile testing was performed with an MTS Insight electromechanical testing system with an environmental bath of 1×DPBS (pH 7.0). Dimensions such as thickness, gage length and width were measured. A special set of grips made from Delrin was tested to avoid corrosion. The samples were then clipped onto the grips which were connected to a 5 N load cell and tested at a strain rate of 1% per second. All the samples were tested until the breaking point. The stress-strain curves were plotted to determine the failure stress, modulus and elongation at the break.

### The Production of the Porous Scaffolding Material

The porous scaffolding material was based on formulating collagen-chitosan biocomposite hydrogels. The mechanical properties of these viscoelastic hydrogels were tailored by varying the concentrations of the components according to the well-known distinct mechanical functions. In addition, the cross-linking condition (e.g., concentration and time) of genipin, a biocompatible cross-linker, was determined by its effects on mechanical properties and cell viability. The fabrication involved molecular self-assembly of SMCs and constituent materials of the biocomposites. Predetermined amount of each component (i.e., collagen, and chitosan) in their solublized ultrapure form was added into buffer solution, neutralized, mixed and degas sed. The pre-polymer solution was injected into a tubular mold consisting of the mandrel with two end caps on an outer tube. Then, the mold was transferred into a cell culture incubator at 37° C. to allow gel formation. After that, the inner mandrel and end caps were ejected from the outer sheath into a sample vial containing medium. Then, the compacted constructs were soaked in a genipin-contained culture medium for cross-linking. Finally, the construct were freeze-dried and incorporate biomolecules such as PDGF.

### Synthesis and Characterization of Nano-Biomaterial for Vascular Tissue Engineering

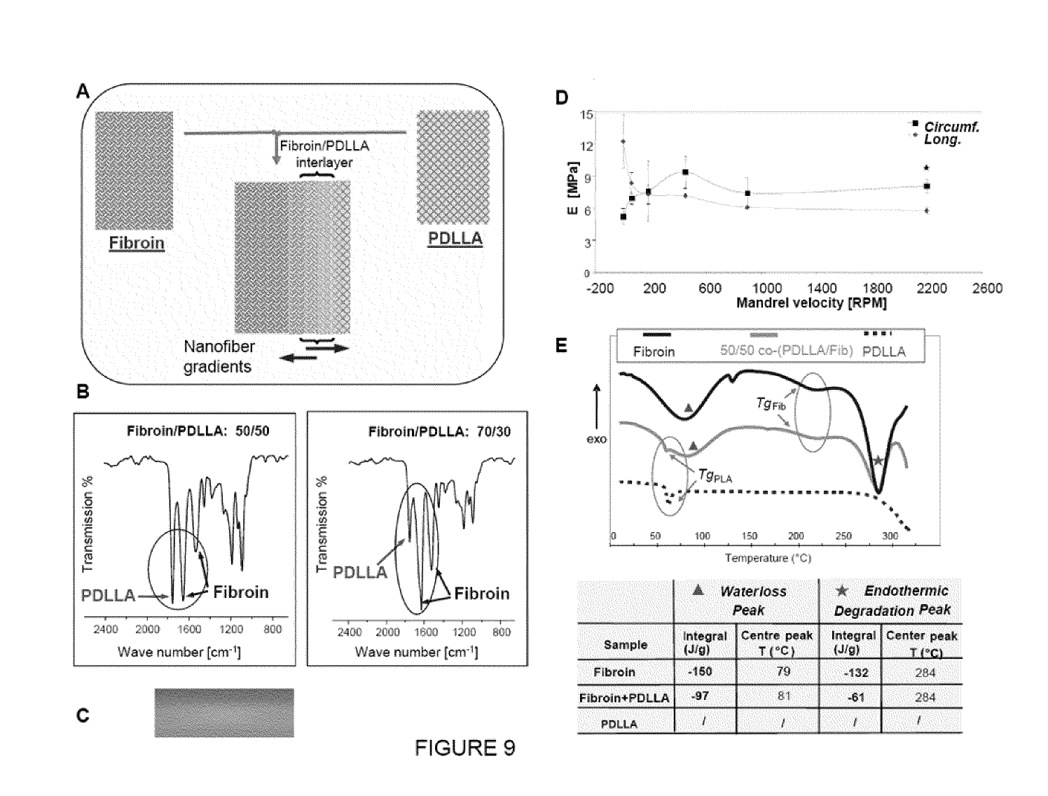

A novel double-electrospinning system has been used to fabricate tubular graft with nanofibrous materials which are composed of ingredient blends with defined nanofiber gradients along the tube wall thickness. Synthetic and/or biological-derived degradable materials such as poly-DL-lactic acid (PDLLA), silk-fibroin and polycaprolactone (PCL), have been electrospun to form constructs with interpenetrating nanofiber gradients. An example of electrospun PDLLA/fibroin nanofiber nets is shown in FIGS. 9A-9E. The nets have been characterized with Fourier transform infrared spectroscopy (FTIR) for composition (FIGS. 7B and 9B), differential scanning calorimetry (DSC) for degradation and hydration, and scanning electron microscope (SEM) for nanostructures. The concentration gradients of fibers and encapsulated molecules are illustrated with fluorescence. The generated ingredient blends of nanofiber gradients using the new double-electrospinning system not only facilitate the formation of molecule gradient to modulate properties spatially and/or temporally, but also provided different surfaces with the one that induced endothelium confluency (PDLLA) facing the lumen (data not shown). Additionally, it was shown that cells readily migrate in the nanofibrous net. It was also noticed that the composition rather than the gradient of the electrospun materials appeared to define the degradation, hydration and other physical properties.

In addition, collagen-chitosan scaffolds have been developed as a structural support for the formation of a vascular media equivalent. In particular, the effects of cross-linking and PDGF-BB (with or without heparin or serum) on biomechanical properties, SMC migration, SMC contraction, and wound healing have been evaluated (FIG. 12). It was found that cross-linking using genipin (a new highly-biocompatible cross-linker) had a significant influence on biomechanical and biological properties of the scaffolds. For example, it enhanced the strength and modulus while inhibited SMC in-growth and contraction. With a low cross-linking condition, the molecule effects of PDGF-BB, a SMC attractant, were studied with or without serum or heparin. It was found that heparin inhibited the PDGF-induced cell migration and contraction while serum did not have any effect. These results show the cross-linked porous scaffold with PDGF molecules serve as mechanically strong and biologically functional scaffold for the formation of a media-equivalent. It also showed that heparin, an anti-coagulant and endothelial growth factor, was localized in a different layer than the porous scaffold.

A non-invasive characterization technique was used to evaluate cell-scaffold-molecule interactions. FIG. 13 shows three-dimensional imaging of sequential events of cell in-growth in a porous biological material-based scaffold. A multi-modal 3-D imaging tool combined with new imaging analysis tools, provided a useful tool to track 3-D cell-scaffold-molecule interactions and analyze the processes non-invasively.

### Pathophysiology of A/V Graft and Evaluation of Drug Efficacy for Graft In Vivo

A/V graft pathology and strategies of drug delivery to prevent hemodialysis vascular access stenosis were examined. The drug delivery platforms included sustained release with biodegradable polymers. Using a porcine model which provides more clinically-relevant pathology than small animals, the dysfunction of ePTFE A/V graft as well as the safety and specificity of anti-proliferative drugs were studied.

A variety of properties and performances of the new graft materials have been successfully demonstrated in vitro. In vivo study is performed with a small animal model (e.g., rabbit) to evaluate application as A/V access with a more clinically-relevant large animal model (i.e., pig) thereafter. Some of the objectives for this study include (1) to the design and evaluate functionality of graft material by performing in vivo grafting on a small animal model and performing ex vivo biomechanical and biological testing; and (2) to perform evaluation of the performances of the graft as A/V access on a porcine model.

### Design and Functionality

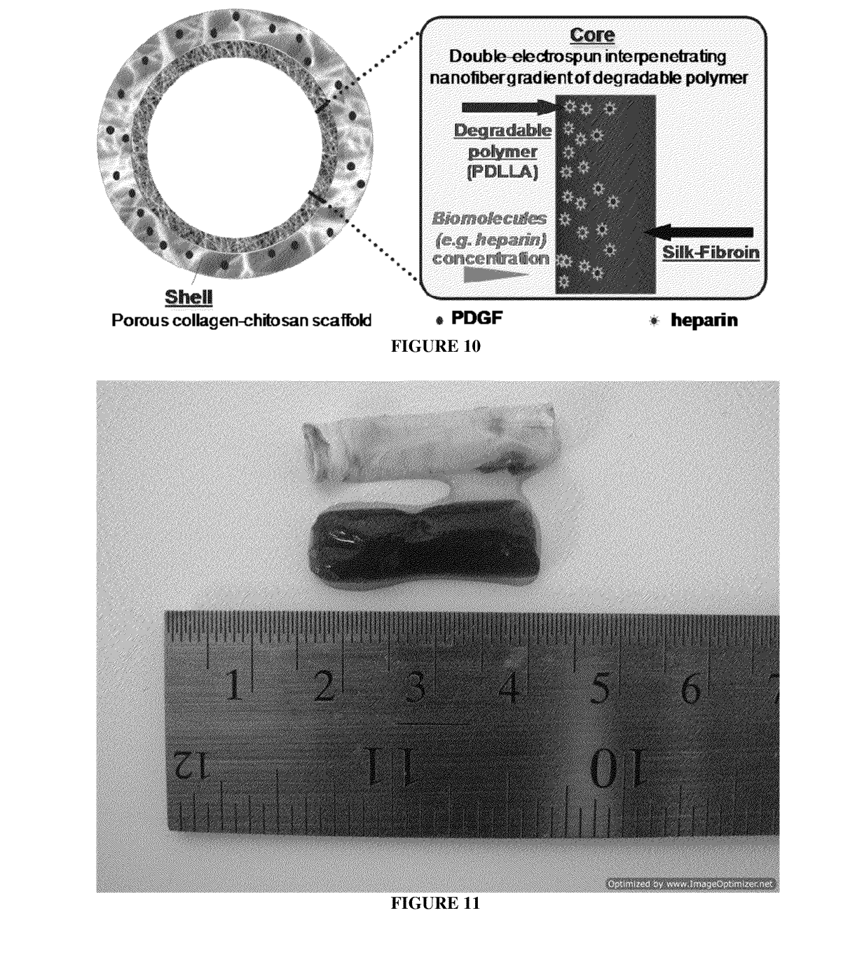

As shown in FIG. 10, each layer of the new bi-layer graft structure contains ECM-like interpenetrating nanofibrous materials as well as localized signaling molecules to control layer-specific cell behaviors. In vitro studies showed fibroin-PDLLA nanocomposite is highly dense, strong and compliant, endothelial-adhesive, and degradable. This latter property can be used to release incorporated biomolecules. It has also been shown that collagen-chitosan nanocomposite with PDGF is highly porous, mechanically stable, and allows SMC in-growth, contraction and migration for wound healing. A rabbit model was chosen for the initial evaluation of the material. The advantages of the rabbit model are that it is a small animal, easy to obtain in pure strain and easy to handle. Furthermore, its clotting system closely resembles that of humans, and rabbit arteries have thromboplastic and fibrinolytic properties similar to those of humans.

Some aspects of the invention integrate mechanical and biological behaviors of the two nanocomposites by forming a bi-layer configuration with an intima-equivalent tubular layer (i.e., core) and a media-equivalent tubular layer (i.e., shell). Through the fabrication technique and the ability to modulate fabrication parameters as disclosed herein, various mechanical, degradable and/or biological properties of the multi-layer graft construct were modified. In some particular embodiments, three parameters were adjusted: (a) degradation rate of the core; (b) heparin molecule loading in the core structure; and (c) PDGF molecule loading in the shell. The material degradation and concentrations of heparin and PDGF are some of the parameters for controlling endothelial function, SMC function and vascular tissue regeneration. When the graft construct has the design parameters changed, a rabbit model was used as a vehicle for investigating vascular remodeling mechanisms and in vivo performance. Time-lapse study is carried out to track the graft performances, e.g., after each pre-determined time period of post-implantation (e.g., 3 days, 15 days, 30 days, 60 days), the animal was euthanized, and the graft was removed for biomechanical, histologic, and immunochemical evaluations.

### Determination of the Optimal PDGF Loading Concentration

The initial loading concentration of PDGF-BB was varied from 0-100 nM in the shell to identify a proper concentration. PDGF plays a role in attracting SMC migration and in-growth, and regulating cell proliferation and stimulating blood vessel growth. Thus, a concentration that balances between its effect of inducing of initial cell in-growth and its effect of preventing cell overgrowth and vessel angiogenesis was needed.

### Determine the Degradation Rate

After determining the PDGF loading concentration, the vascular remodeling of media equivalent (i.e., shell) was controlled. The next step was to test the influence of physical properties of intimal equivalent (i.e., core) on the changes of biomechanical and biological properties of the vascular graft construct. The degradation of shell was coordinated with vascular tissue regeneration and matrix production in the core. Therefore, one of the design variables is the degradation rate, striking a balance between graft strength, compliance and vascular cell interactions. The shell started as a highly-dense, strong material incorporating heparin gradient. It was desirable to seek a compromise between an intimal-equivalent having very slow degradation, being mechanically robust but offering limited release of heparin, and limited diffusion of signaling molecules between vascular cells after a long period of post-implantation, and an intimal-equivalent having fast degradation to release the biomolecules and diffusion of signaling molecules between cells but mechanically weakened in a short post-implantation period before media-equivalent becomes mechanically strong. The degradation rate of the shell was used to control the temporal profile of mechanical strength and compliance of the graft, as well as the behaviors and interactions of EC and SMC.

### Determining the Heparin Loading Concentration

Heparin is an important molecule that prevents coagulation in the lumen, and regulates endothelial and SMC function. It enhances endothelial growth while inhibits SMC growth and migration. Therefore, it is believed to play a role in the short-term and long-term graft patency. Injection of heparin usually is not an efficient way for local delivery. One of the unique features of the present invention is the creation of a gradient distribution of heparin. This is desirable as heparin is likely in great need right after the grafting procedure to repair vessels that have been injured during the grafting procedure. Heparin is believed to be required to prevent clotting and enhance endothelial growth and function. But its role decrease after the formation of a functional endothelium. Excessive heparin doses can cause other cardiovascular problems. The initial loading concentration of heparin was varied from 0 to about 100 M in the core structure to evaluate the proper concentration.

### Construct Fabrication and Processing

The design of one particular embodiment of the bionanocomposite material of the invention is schematically shown in FIG. 10. The engineered constructs were made up of two coaxial layers of bionanocomposites: the core composed of fibroin-PDLLA composites and the shell composed of self-assembled collagen-chitosan composites. Collagen-chitosan composites, both naturally-derived materials, were cross-linked for mechanical stability and to prevent rapid degradation in vivo. The composites form 3-dimensional porous coating around the intima-equivalent. In some embodiments, the bionanocomposite materials included molecular sequences that facilitate cell in-growth and remodeling, thereby inducing in-growth of smooth muscle cell (SMC) when grafted. Fibroin seeding of functional endothelial cell (EC) (e.g., to form a non-thrombogenic surface) is a slowly-degradable biological material isolated from silk while PCL is a highly-biocompatible, slowly-biodegradable synthetic material. The fibroin-PCL nanofiber shell can form a strong and highly-dense wrap around porous gel core to provide required mechanical properties at the time of implantation. In addition, the novel design of the mulitilayer bionanocomposite also included layer-specific biomolecules immobilized in the constructs. Biomolecules such as heparin which can attract EC and regulate cell proliferation were immobilized by forming complexes with chitosan in the core; while biomolecules such as TGF-β which can promote SMC differentiation and self-healing process were embedded in fibroin nanofibers in the shell. Thus, the release of cell-specific molecules can be spatially defined in each layer. The fabrication techniques involved two steps: (1) double-electrospinning fibroin and PCL nanofibers on a rotational mandrel; and (2) developing self-assembled collagen-chitosan composite gel surrounding the tubular structure of PCL/PDLLA fiber. The incorporation of heparin in collagen-chitosan gels used a joint precipitation process of cationic chitosan and anionic heparin in the form of polymeric nanoclusters caused by the addition of a cross-linking agent such as sodium tripolyphosphate. A novel electrospinning (e.g., double-electrospinning) technique was used to prepare a gradient distribution of TGF-β through the gradient blend of nanofibers in the composites. TGF-β molecules were added into the fibroin solution before electrospinning.

### Grafting on a Rabbit Model

New Zealand White rabbits weighing between 3 and 4 kg are used randomly in this study. All animals are anesthetized with ketamine (70 mg/kg) intramuscularly, incubated with a 3-mm cuffed endotracheal tube, and mechanically ventilated with a pressure-controlled ventilator. An adequate level of anesthesia was maintained by inhaled isoflurane (1%-3%). A limb-lead electrocardiogram is monitored. A central ear artery catheter is inserted to continuously monitor systemic arterial pressure. Arterial blood samples are drawn every 30 minutes to determine arterial oxygen tension, acid-base balance, and electrolyte levels. Ringer's lactate solution is infused continuously, and sodium bicarbonate, potassium chloride, and calcium chloride are supplemented to maintain pH and electrolytes within normal values. Enrofloxacin (5 mg/kg) is administrated preoperatively to reduce the risk of infection. After a midline abdominal incision, the intestines are displaced to the right side and covered with moistened gauze. The infrarenal aorta is carefully dissected from the surrounding tissue. The lumbar arterial branches are spared to avoid spinal cord ischemia. Intravenous heparin (200 U/kg) is administered. The abdominal aorta is clamped with microapproximator clamps between the lumbar branches and transected. The nanomaterial or ePTFE grafts are anastomosed to the aorta in an end-to-end fashion with a continuous 7-0 polypropylene suture. The total anastomotic time is less than 30 minutes in every animal (22±4 minutes). Blood flow is measured with an ultrasonic flow probe (Transonic System Inc, Ithaca, N.Y.) proximally and distally. The abdominal incision is closed. The animals are administered with analgesia (buprenorphine 0.3-0.5 mg/kg) and antibiotic (enrofloxacin 5 mg/kg) treatments subcutaneously twice daily for 2 days after surgery. Postoperative antiplatelet therapy (aspirin 15 mg/kg) is administered daily. At 1 and 3 months after surgery, the animals are anesthetized again with intramuscular ketamine (70 mg/kg). The abdominal incision is reopened. The surgical site is examined for adhesions, fibrosis, hematoma, or arteriovenous fistula. Blood flow at the proximal and distal anastomoses is measured with an ultrasonic flow probe. Six weeks after graft placement, the animals are euthanized, and the graft is carefully removed for biomechanical, histological, and immunohistochemical evaluations. Blocks of grafts, vessels and graft/vessel anastomosis are stained with H&E, Masson's trichrome stain, EVG which stains elastic lamina black, collagen red and the remainder yellow. Fresh thrombi, when present, are discernible from hyperplasia by their distinctive red color and absence of SMCs. They are separately measured and not included in hyperplasia cross-sectional area. The I/M ratio (ratio of intimal thickness to medial thickness), the hyperplasia-to-graft (H/G) ratio, and visual scoring are carried out by ranking the amount of hyperplasia formed on a scale of 0-5 (0=no hyperplasia and 5=lumen completely occluded by hyperplasia, not fresh thrombosis) with 0.5 increments of scale. Additionally, specific cell markers for ECs and SMCs are applied to analyze the cell components in the hyperplasia.

### Ex Vivo Evaluation of Material and Mechanical Properties

Before implantation, physical and biomechanical properties, including tensile strength/elasticity (including longitudinal and circumferential tensile strength/elasticity), burst strength, suture retention, permeability and compliance were performed according to ISO 7198 standard (Cardiovascular implants-tubular vascular prostheses). The materials are characterized with SEM (structure) and FTIR (chemistry). After implantation, the grafts removed from rabbits are tested for tensile strength/elasticity, burst strength and compliance tests.

### Evaluation of the Optimized Graft for A/V Access Application

To demonstrate A/V access performance and advantage over ePTFE, a bionanocomposite material of the present invention was tested on a clinically-relevant animal model. In selecting the appropriate animal model, efforts were made to evaluate device performance in models that would demonstrate performance attributes in a clinically meaningful fashion. The porcine model was used to assess the following aspects of the graft: (1) the capability of the graft to remodel and form into an arterial-like structure; and (2) provide long-term (e.g., 6 months) patency with minimal intima hyperplasia, stenosis and thrombosis.

A bionanocomposite material was fabricated with the design, structure and composition for the evaluation tests. The ePTFE vascular grafts were used as a control. In conducting the preclinical animal studies for the material, the same introduction method that is used in the clinical setting was modeled as much as possible.

### Biomimetic Vascular Grafts

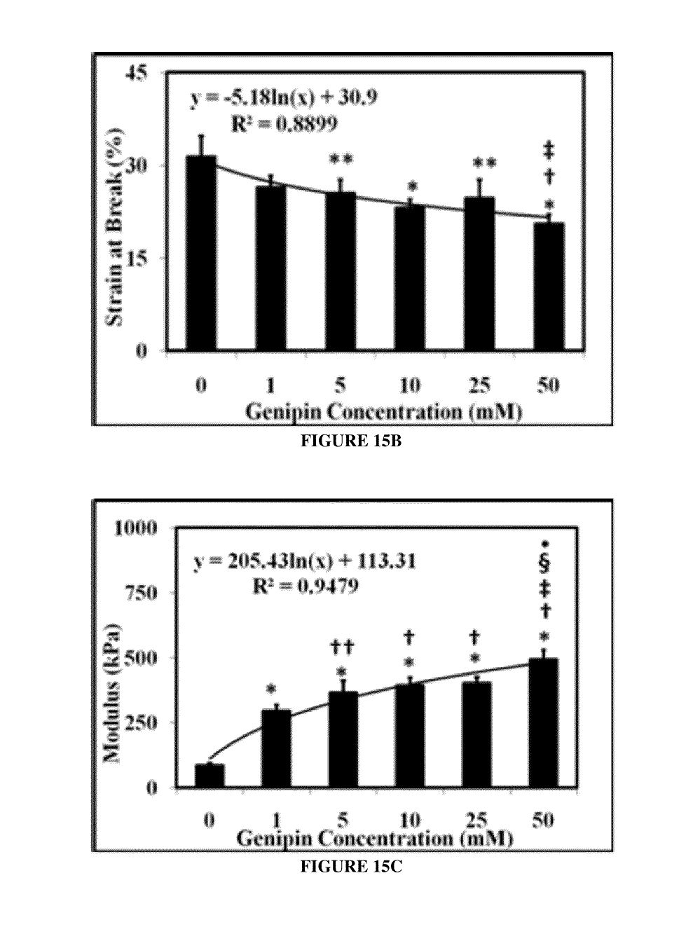

The vascular media, a layer of the blood vessel wall containing smooth muscle cell (SMC), is often the target functional tissue in the construction of biomimetic vascular grafts. It has a major contribution to mechanical properties and biological functions of vessels including contraction and healing. The present inventors have studied the effects of crosslinking and biomolecule conditions in the development of mechanically strong and stable, biologically functional constructs for vascular media equivalents. To this end, genipin was used to crosslink collagen-chitosan-elastin (CCE) constructs, and the effects of genipin concentration on the mechanical properties and SMC activity of CCE gel constructs were examined. Results revealed that mechanical strength and stiffness of CCE constructs significantly increased with genipin crosslinking and gradually increased with the increase of genipin concentration from 1 mM to 25 mM (FIG. 15), but the crosslinking significantly inhibited SMC contraction and invasion in the CCE gels. No SMC contraction or invasion was observed in the gels crosslinked with genipin at a concentration of 5 mM or above. Results from ATR-FTIR characterizations showed that crosslinking changed several functional groups and small differences existed among the samples crosslinked with different concentrations of genipin.

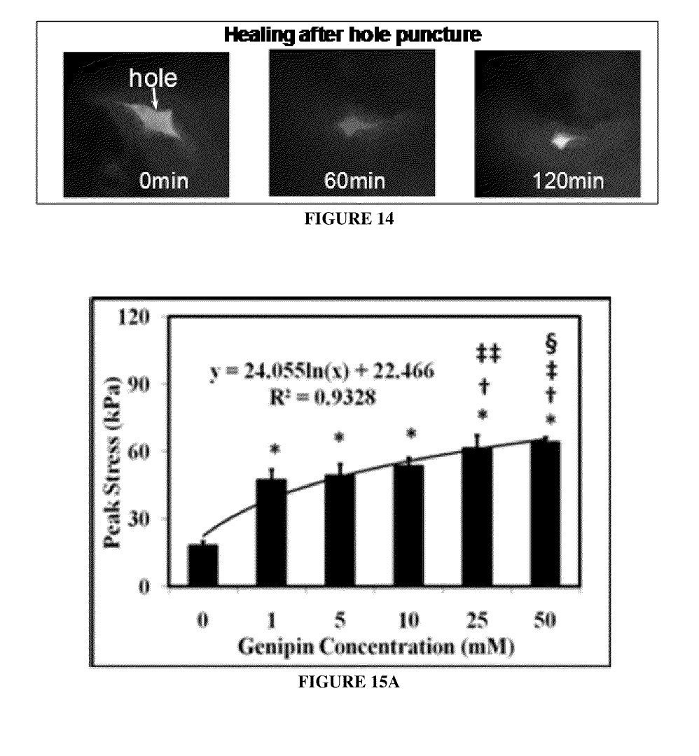

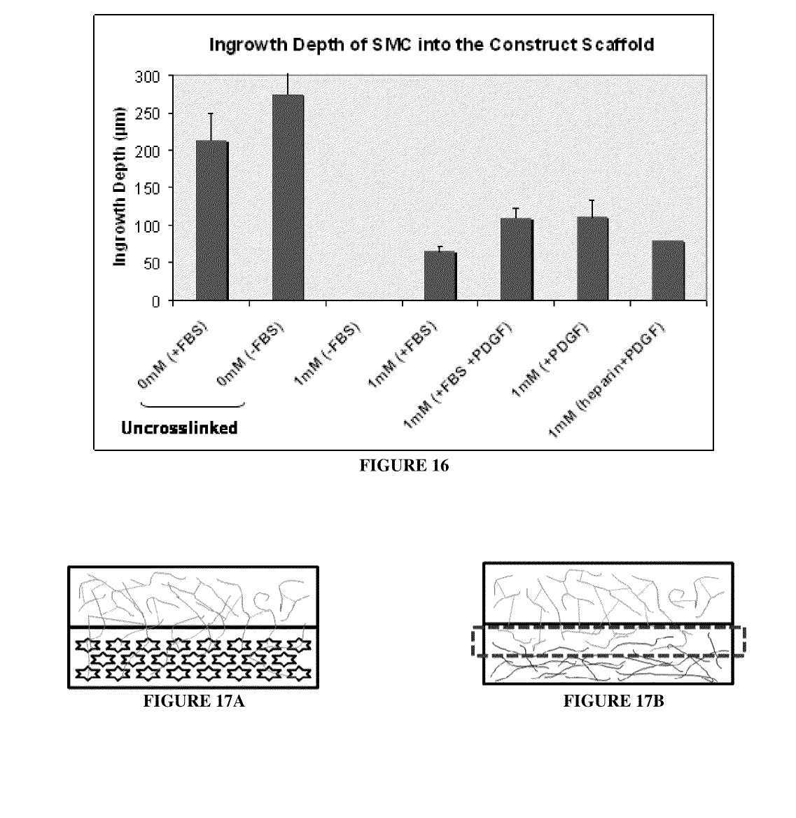

To enhance biological activities of cells on the genipin-treated CCE constructs, soluble molecule factors were incorporated with structural constructs and their effects on SMC activities were evaluated. These conditions include heparin, platelet-derived transforming growth factor (PDGF), high-concentrated fetal bovine serum (h-FBS), mixture of heparin and PDGF and mixture of h-FBS and PDGF. Results showed that h-FBS and PDGF synergistically stimulated SMC invasion in and contraction of genipin-crosslinked CCE (FIGS. 13 and 16). Though heparin is often incorporated on the vascular graft to prevent thrombosis and is known to stimulate endothelial growth, it was shown to have inhibitory effects on PDGF-induced SMC invasion into the crosslinked constructs. Results indicated that for the design of an engineered vascular construct, heparin should be localized on the luminal surface or the intima equivalent of the construct, instead of being uniformly distributed throughout the construct or being presented in the media equivalent as it prevent the constructs from inhibiting SMC growth, invasion and remodeling during the initial stage of implantation. The ingrowth of SMC in the vascular media equivalent also promoted wound healing process of the construct scaffold by showing the closure of the puncture hole (FIG. 14). These results indicate that designing vascular media equivalent to encourage tissue regeneration should employ an appropriate combination of crosslinking condition and soluble biomolecule factors to strike an appropriate balance between mechanical properties and biological functions.

### Improved Adhesion Between COL-CHI-ELN and PCL Layers

Note: The blue lines in FIGS. 17A and 17B represent collagen fibers, the orange lines in FIGS. 17A and 17B represent the genipin crosslinking, the stars in FIG. 17A represent the increase number of pores in the top few layer of PCL and the black lines in FIG. 17B represent the PCL fibers. The dotted box in FIG. 17B indicates the change of chemical composition of top layer of PCL with electrospraying or double-electrospinning.

### Increasing Porosity Near Top Surface of PCL:

The PCL fibers were electrospun on to the cylindrical mandrel for about 90-100 μm. Afterwards the PCL was double electrospun with polyethylene oxide (PEO) for about 25 μm on top of the pure PCL layer. Then the PEO was dissolved in deionized water. The PEO dissolved and the porosity of the top layers of PCL was increased. This increase in porosity allowed the movement of the COL-CHI-ELN pre-gelation mixture to enter the PCL matrix, around the PCL fibers, thereby ensuring a stronger adhesion between the two layers. This is schematically represented in FIG. 17A and the result is shown in FIG. 11.

Changing PCL Top Layer Chemical Composition by Electrospraying with Collagen:

First, the PCL fibers were electrospun on to the cylindrical mandrel for about 90-100 μm. Then, for about 25 μm, the collagen was electrosprayed while electrospinning the PCL. When this PCL tube electrosprayed with collagen was used for synthesizing the bilayer with the genipin-added COL-CHI-ELN mixture, genipin crosslinked collagen, chitosan and elastin between themselves and with the collagen present on the PCL layers at the interface, thereby forming a strong covalent hemiaminal bond. This also ensured proper adhesion between the two layers. This is represented in FIG. 17B.

Changing PCL Top Layer Chemical Composition by Double Electrospinning with Collagen:

First, the PCL fibers were electrospun on to the cylindrical mandrel for about 90-100 μm. Then, for about 25 μm, the PCL was double electrospun with collagen. When this PCL tube double electrospun with collagen was used for synthesizing the bilayer with the genipin-added COL-CHI-ELN mixture, genipin crosslinked collagen, chitosan and elastin between themselves and with the electrospun collagen fibers present along with the PCL near the interface, thereby forming a strong covalent hemiaminal bond. This also ensured proper adhesion between the two layers. This is represented in FIG. 17B.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed:

1. A bionanocomposite material comprising at least two coaxial layers of bionanocomposites and having an interior that is capable of allowing a fluid to flow therethrough, said bionanocomposite material comprising:
a core structure; and
a shell structure encapsulating said core structure,
wherein one of said core structure or said shell structure comprises a biocompatible natural-derived material and the other comprises a biocompatible biomimetic nanostructure.

2. The bionanocomposite material of claim 1, wherein said bionanocomposite material is tube-like or in a tubular form.

3. The bionanocomposite material of claim 1, wherein said core structure comprises a porous biocompatible natural-derived material and said shell structure comprises said biocompatible biomimetic nanostructure.

4. The bionanocomposite material of claim 1, wherein said shell structure comprises a porous biocompatible natural-derived material and said core structure comprises said biocompatible biomimetic nanostructure.

5. The bionanocomposite material of claim 1, wherein said biocompatible natural-derived material is biodegradable.

6. The bionanocomposite material of claim 5, wherein said biocompatible natural-derived material comprises a biodegradable natural-derived composite hydrogel.

7. The bionanocomposite material of claim 6, wherein said biodegradable natural-derived composite hydrogel comprises collagen, chitosan, elastin, or a combination thereof.

8. The bionanocomposite material of claim 1, wherein said core structure further comprises vascular smooth muscle cell, a bioactive material, or a combination thereof.

9. The bionanocomposite material of claim 1, wherein said core structure is capable of facilitating cell adhesion, in-growth, remodeling, or a combination thereof.

10. The bionanocomposite material of claim 1, wherein said biocompatible biomimetic nanostructure comprises fibroin, collagen, a biodegradable polymer, or a combination thereof or a derivative thereof.

11. The bionanocomposite material of claim 1, wherein said shell structure further comprises a signaling compound that is capable of providing long-term remodeling signal in vivo.

12. The bionanocomposite material of claim 1, wherein said bionanocomposite material has an average burst strength ranging from about 1200 mmHg to about 2400 mmHg.

13. The bionanocomposite material of claim 1, wherein said bionanocomposite material has an average modulus of from about 3 MPa to about 15 MPa.

14. The bionanocomposite material of claim 1, wherein said core structure comprises a core additive substance that promotes endothelial cell adhesion or regulates cell proliferation, or both.

15. The bionanocomposite material of claim 15, wherein said core additive substance comprises heparin.

16. The bionanocomposite material of claim 1, wherein said shell structure comprises a shell additive substance that promotes smooth muscle cell differentiation, self-healing process, or a combination thereof.

17. The bionanocomposite material of claim 17, wherein said shell additive substance comprises TGF-β.

18. A method for producing a tubular bionanocomposite material comprising at least two coaxial layers of bionanocomposites, said method comprising:
placing a core structure comprising a porous biocompatible natural-derived material on a mandrel; and
double-electrospinning at least two different biocompatible shell structure materials on to the core structure to form a shell structure comprising a biocompatible biomimetic nanostructure that encapsulates the core structure, whereby a tubular bionanocomposite material comprising at least two coaxial layers of bionanocomposites with a porous core structure is produced.

19. The method of claim 16, wherein said step of double-electrospinning at least two different biocompatible shell structure materials is simultaneously applied to the moving core structure on the mandrel to produce a substantially intertwined shell nanostructure.

20. The method of claim 16, wherein the core structure is produced by a self-assembly process.
